(19) United States
(12) Patent Application Publication
(10) Pub. No.: US 2005/0131051 A1
(43) Pub. Date: Jun. 16, 2005

(54) 2-3-disubstituted quinuclidiness as modulators of monoamine transporters and theraperutic and diagnostic methods based thereon

(76) Inventors: Shaomeng Wang, Ann Arbor, MI (US); Sukumar Sakamuri, Plainsboro, NJ (US); Istvan Enyedey, Hamden, CT (US); Alan Kozikowski, Princeton, NJ (US)

Correspondence Address: PILLSBURY WINTHROP SHAW PITTMAN, LLP, P.O. BOX 10500, MCLEAN, VA, 22102, US

(21) Appl. No.: 10/362,164

(22) PCT Filed: Aug. 21, 2001
(86) PCT No.: PCT/US01/25991

Related U.S. Application Data
(60) Provisional application No. 60/226,581, filed on Aug. 21, 2000.

(51) Int. Cl. A61K 31/407; C07D 487/04

(52) U.S. Cl. 514/412; 548/453

(57) ABSTRACT

The present invention relates to a class of compounds of formula (I) and (II): 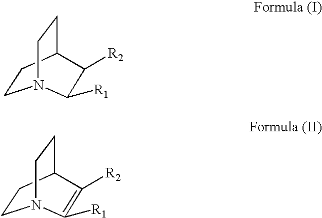
wherein R₁ is hydrogen; linear or branched C₁-C₁₅ alkyl; C₁-C₁₅ alkenyl; C₃-C₆ cycloalkyl; mono, di, tri, tetra, penta substituted aryl or heteroaryl; COOR₃; —(CH₂)ₙ-aryl; —COO—(CH₂)ₙR₃; —(CH₂)ₙ—COOR₃; —C(O)R₃; —C(O)NHR₃; or an unsubstituted or substituted oxadiazole; and R₂ is hydrogen; linear or branched C₁-C₁₅ alkyl; C₁-C₁₅ alkenyl; C₃-C₆ cycloalkyl; mono, di, tri, tetra, penta substituted aryl or heteroaryl; unsubstituted or substituted naphthyl; 1,3-Benzodioxole; fluorene; indole; isoquinoline; quinoline; pyridine; pyrimidine; onnthracene; or —(CH₂)ₙ-Ph; wherein the heteroaryl comprises N, O, or S, the mono or multi substituents on the aryl or heteroaryl are independently C₁-C₅ alkyl, C₁-C₅ alkenyl, H, F, Cl, Br, I, —NO2, NHR, or —OR, R is C₁-C₇ alkyl; R₃ is C1-C5 alkyl, C1-C5 alkenyl, benzyl, substituted aryl or heteroaryl; and n=1-7. These compounds are discovered, synthesized and confirmed as potent inhibitors of dopamine (DA), serotonin (5-HT), and norepinephrine inhibitors. These compounds are therefore particularly useful in the treatment conditions or diseases wherein modulation of the monoamine neurotransmitter system involving dopamine (DA), serotonin (5-HT), and norepinephrine plays a role.

### BACKGROUND OF THE INVENTION

1. Related Applications

This application is based on U.S. Provisional Application Ser. No. 60/226,581, filed Aug. 21, 2000, the contents of which are hereby incorporated by reference in their entirety.

2. Field of the Invention

The present invention relates to discovery, synthesis and enantiomer separation of compounds 2,3-disubstituted quinuclidines as potent inhibitors for dopamine, serotonin and norepinephrine transporters and therapeutic uses of such compounds.

3. Summary of the Related Art

The specific reuptake of the monoamine neurotransmitters, dopamine (DA), serotonin (5-HT), and norepinephrine (NE) from the synaptic cleft is the primary physiological mechanism for the termination of monoaminergic neurotransmission. Blocking the uptake increases synaptic availability of the neurotransmitters, thereby potentiating the signal (Kitayama, S. Dohi, T. Jpn. Pharmacol. 1996, 72, 195-208). This has been exploited to develop treatments for a large number of neurological disorders. The selective serotonin transporter (SERT) inhibitor, such as fluoxetine (Prozac) is used for the treatment of depression. The selective dopamine transporter (DAT) inhibitor, benzotropine, is used clinically for the treatment of Parkinson's disease. Other potent and selective DAT inhibitors such as RTI-113 and GBR 12909 are now in clinical trials for the treatment of cocaine abuse. Norepinephrine transporter (NET) inhibitors such as desipramine are effective in the treatment of depression. The present invention relates to a novel class of compounds, 2,3-disubstituted quinuclidines as potent inhibitors of dopamine, serotonin and norepinephrine transporters and their therapeutic use.

Potent, long-duration DAT inhibitors with no or little abuse liability themselves can be used for the treatment of cocaine abuse. One aspect of the present invention can be used as novel therapeutic agents for the treatment of cocaine abuse. Cocaine abuse is one of the greatest concerns of the American public today, and has therefore become a focus of medical, social, and political debate. Cocaine is one of the most addictive substances known, and cocaine addicts may lose their ability to function at work or in interpersonal situations. Although cocaine potently inhibits the reuptake of both norepinephrine (NE) and serotonin (5-HT), many lines of evidence indicate that its ability to act as a reinforcer stems from its ability to inhibit the reuptake of dopamine (DA) into dopaminergic neurons. Cocaine exerts this effect via specific interaction with DA transporter (DAT) proteins (cocaine receptor) located on DA nerve terminals. This increase of dopaminergic transmission in the reward mediating brain mesolimbic system is the essence of the dopamine hypothesis for cocaine action.

However, recent studies have shown that the simultaneous flow of dopamine, serotonin and norepinephrine plays an important role in the molecular mechanisms involved in addiction to cocaine. A common molecular aspect to the flow of dopamine, serotonin and norepinephrine involves monoamine transporters. Therefore, it would be greatly beneficial if a class of small molecule compounds could be identified or designed to modulate the activity of monoamine transporters, thereby simultaneously modulating the uptake of dopamine serotonin and norepinephrine by monoamine transporters. Such novel compounds and therapeutic and diagnostic methods based thereon will be greatly beneficial in the treatment of numerous neurological disorders. Of particular interest are lead compounds capable of antagonizing all or some of cocaine action.

### SUMMARY AND OBJECTS OF TILE INVENTION

It is an object of the invention to provide compounds which inhibit abnormal dopamine signaling in the synaptic space in neurons.

It is another object of the invention to provide compounds which are antagonistic of cocaine.

Another object of the invention is to provide a method for modulation of brain dopamine flow in a subject in need of such control. The method comprises administering to the subject a compound identified according to the above-described method.

Yet another object of the invention is to provide a method of inhibiting cocaine action in a subject in need of such inhibition comprising administering to the subject a compound identified according to the method described above.

A still further object of the invention is to provide a method of promoting dopamine reuptake action in a subject in need of such action comprising administering to said subject a compound identified according to the method described above.

In one aspect, the invention provides a compound or a pharmaceutically acceptable salt thereof, wherein the compound is of formulae (I) or (II): 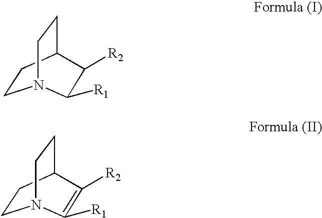
wherein R₁ is a hydrogen; linear or branched C₁-C₁₅ alkyl; C₁-C₁₅ alkenyl; C₃-C₆ cycloalkyl; mono, di, tri, tetra or penta substituted aryl or heteroaryl; —(CH₂)ₙ-aryl; COOR₃; —COO—(CH₂)ₙR₃; —(CH₂)ₙ—COOR₃; —C(O)R₃; —C(O)NHR₃; or an unsubstituted or substituted oxadiazole; R₂ is a hydrogen; linear or branched C₁-C₁₅ alkyl; C₁-C₁₅ alkenyl; C₃-C₆ cycloalkyl; mono, di, tri, tetra or penta substituted aryl or heteroaryl; unsubstituted or substituted naphthyl; 1,3-Benzodioxole; fluorene; indole; isoquinoline; quinoline; pyridine; pyrimidine; anthracene; or —(CH₂)ₙ-Ph; and R₃ is C₁-C₅alkyl, C₁-C₅ alkenyl, benzyl, substituted aryl or heteroaryl; and n=1-7; and wherein the heteroaryl comprises N, O, or S, the mono or multi substituents on the aryl or heteroaryl are independently C₁-C₅ alkyl, C₁-C₅ alkenyl, H, F, Cl, Br, I, —NO₂, NHR, or —OR, wherein R is C₁-C₇ alkyl.

The compounds of formula (I) are preferably prepared and isolated in an enantiomeric form selected from the group consisting of the (±)-; (+)- and (⁻) isomers.

Another aspect of the invention provides a method of preparing a compound according to the invention, wherein the method comprises:(a) preparing a quinuclidinone having a first substituent under Mannich reaction conditions; and (b) reacting the product of step (a) to add a second substituent to the quinuclidinone thereby producing the compound. The method of the invention, further comprises (c) reducing the compound obtained in step (b) to produce a disubstituted quinuclidine of formula (I).

The invention also provides a method of treatment of a condition or disease wherein dopamine flow in the brain plays a role, wherein the method comprises administering to a subject in need of such treatment an effective amount of a compound of formulae (I) or (II) as described above.

The invention also provides a method of treatment of a condition or disease wherein serotonin flow plays a role, wherein the method comprises administering to a subject in need of such treatment an effective amount of a compound of formulae (I) or (II) as described above.

The invention also provides a method of treatment of a condition or disease wherein norepinephrine flow in the brain plays a role, wherein the method comprises administering to a subject in need of such treatment an effective amount of a compound of formulae (I) or (II) as described above.

One particularly advantageous aspect of the invention provides a method for the treatment of cocaine abuse in a subject in need of such treatment, wherein the method comprises modulating at least one of dopamine, serotonin and norepinephrine monoamine transmitter reuptake by administering to said subject a compound of formulae (I) or (II) as described above. The compounds of the invention are greatly advantageous in the treatment of various neurological disorders that involve the dopamine, serotonin and/or norepinephrine monoamine transmitter reuptake. The compounds of the invention are particularly useful in the treatment of condition such as clinical depression, anxiety, Alcoholism, eating disorders and Parkinson's disease.

The compounds of the invention are also useful in the treatment of chronic pain and obsessive compulsive disorders by modulating at least one of dopamine, serotonin and norepinephrine monoamine transmitter reuptake by administering to a subject a compound according of formulae (I) or (II).

Preferred compounds according to the invention include 2-Butyl-3-phenylquinuclidine, preferably in substantially pure (±)- enantiomeric form, and 2-Butyl-3-(4-methylphenyl)quinuclidine, preferably in substantially pure (±)- or (+)- enantiomeric form.

Other preferred compounds of the invention are listed in Table 2.

In another aspect, the invention provides a method of diagnosis of a condition wherein modulation at least one of dopamine, serotonin and norepinephrine monoamine transmitter reuptake plays a role, the method comprising contacting a sample of body fluid with a compound of formulae (I) or (II), wherein the compound is labeled. Preferred labeling agents include radioactive agents, fluorescent agents and labeling agents containing a traceable electromagnetic moiety.

### BRIEF DESCRIPTION OF THE TABLES AND DRAWINGS

Table 1 is representative monoamine transporter inhibitors of Formula (I) and their activity at the three monoamine transporter sites.

Table 2 is representative monoamine transporter inhibitors of Formula (II) and their activity at the three monoamine transporter sites.

FIG. 1 is the chemical structures of cocaine, WIN 35065-2, the lead compound (3) and a potent cocaine analog.

FIG. 2. is the pharmacophore model used in 3D-database pharmacophore searching, which led to the identification of the lead compound 3.

FIG. 3 is the two possible overlaps between the lead compound 3 (green) and cocaine (yellow) using the three pharmacophore elements defined in FIG. 2.

FIG. 4 is an alternative overlap between the lead compound 3 (green) and cocaine (1, yellow) using an augmented pharmacophore model.

FIG. 5 is the overlaps between the designed analog 12 (green) and cocaine (yellow) (A), and between 12 (green) and WIN 35065-2 (2, yellow) (B).

FIG. 6 is the X-ray structure of analog 13.

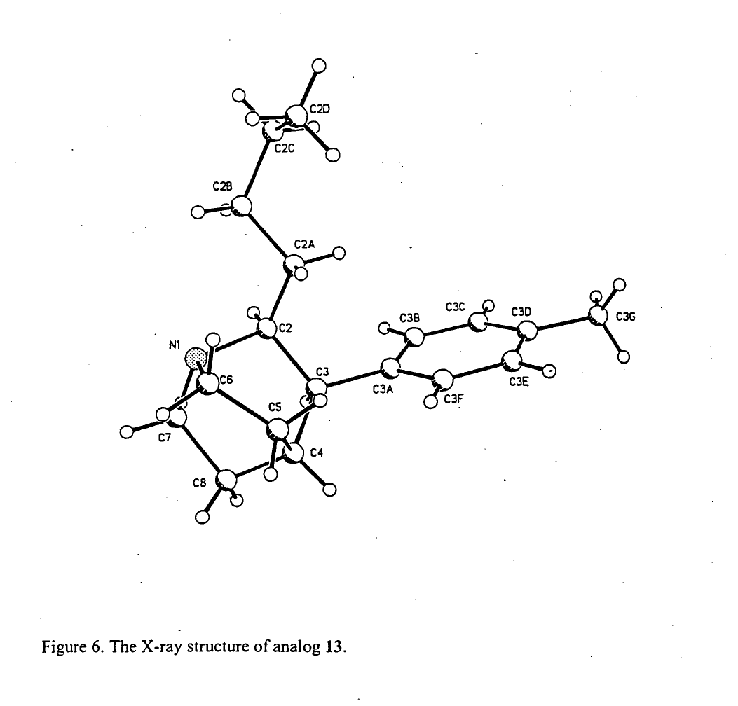

FIG. 7 shows scheme I which illustrates the synthesis route of compounds with general formulae (I) and (II).

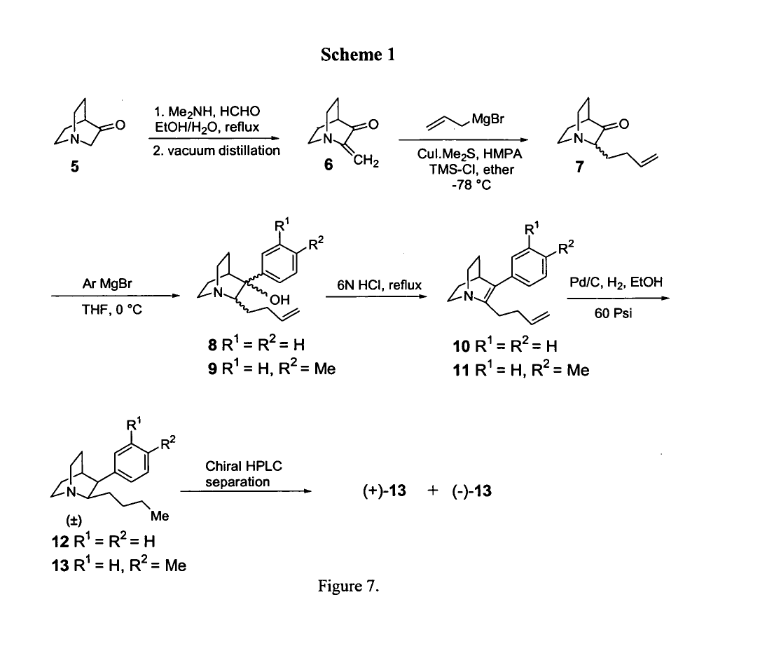

### DETAILED DESCRIPTION OF THE PREFERRED EMBODIMENTS

A lead compound according to the invention is a chemical compound selected for chemical modification to design analog compounds useful in the treatment of a given condition. The lead compound can be a known compound or a compound designed de novo.

A pharmacophore according to the invention is a chemical motif including a number of binding elements. The elements are presumed to play a role in the activity of compounds to be identified as a lead compound. The pharmacophore will be defined by the chemical nature of the binding elements as well as the geometric arrangement of those elements.

Basically, our invention is applicable to conditions or diseases where modulation of the monoamine neurotransmitter system involving dopamine (DA), serotonin (5-HT), and norepinephrine, may have beneficial effects or diseases where modulation of the monoamine neurotransmitter system involving dopamine (DA), serotonin (5-HT), and norepinephrine, may have beneficial effects. Examples of such conditions include depression anxiety alcoholism chronic pain eating disorder obsessive compulsive disorders cocaine abuse.

The present invention includes compounds which are rationally designed to control dopamine flow in the brain. These compounds can be dopamine transporter inhibitors and/or cocaine antagonists. Rational design of the compounds of the present invention includes identifying a mechanism associated with dopamine flow in the brain. Information relating to the mechanism is then analyzed such that compound structures having possible activity in interfering with such a mechanism are formulated. In particular, structures are synthesized based on “building blocks”, wherein each building block has a feature potentially capable of interfering with a particular mechanism associated with dopamine flow, particularly, a mechanism mediated by dopamine transporter protein (DAT).

Compounds having different building block combinations are then synthesized and their activity in relation to the identified mechanism tested. Such tests are conducted in vitro and/or in vivo. The information obtained through such tests is then incorporated in a new cycle of rational drug design. The design-synthesis-testing cycle is repeated until a candidate compound having the desired properties for a targeted therapy; e.g. dopamine flow control, is obtained. The candidate compound is then clinically tested.

An approach for controlling dopamine flow in the brain for the treatment of cocaine addiction is to design cocaine antagonists which can affect dopamine uptake. More specifically, this approach is based on rationally designing compounds which are antagonists of cocaine which reduce or block cocaine binding to DAT. Preferably, antagonists are designed which reduce or block cell cocaine binding while leaving other aspects of dopamine transport unaffected. The designed antagonists should provide a basis for therapeutic protocols based on the selective control of dopamine transport and thereby control of synaptic signaling without disruption of the normal flow of dopamine in the brain.

Although both cocaine and dopamine bind to the DAT, recent mutagenesis and pharmacokinetic studies provide evidence that dopamine and cocaine do not share an identical binding site on the DAT. Thus, one object of the present invention is to discover molecules that will compete with cocaine at its binding site, yet bind to the DAT in a manner that would not significantly inhibit the transport of dopamine. These molecules could potentially function as cocaine antagonists or as partial agonists if they bind in such a way that inhibition of dopamine uptake is incomplete. Such compounds would be useful to counter some of the adverse effects of cocaine in cases of cocaine overdose or help maintain patients in cocaine treatment program.

Recent advances in molecular biology have identified the amino acid sequences of the DAT, but no experimental 3D structures have been obtained for the DAT. The lack of experimental structures makes it difficult to employ a structure-based design strategy for the discovery of DAT inhibitors as cocaine antagonists.

On the other hand, a wealth of SAR data on cocaine analogs and other classes of dopamine transporter inhibitors are available. This makes it feasible to derive “putative 3D pharmacophore models”, defined as the representation of crucial chemical structural features and their 3D geometric relationships that are important for the biological activity of interest. With the pharmacophore models, one can search large chemical databases to discover compounds whose 3D structures meet the pharmacophore requirements.

Using a lead compound identified according to the invention, a large number of DA inhibitors were designed and tested. Compounds have been identified which exhibit promising cocaine antagonism in our functional assay.

Identification of a Pharmacophore for Rational Drug Design of Cocaine Antagonists

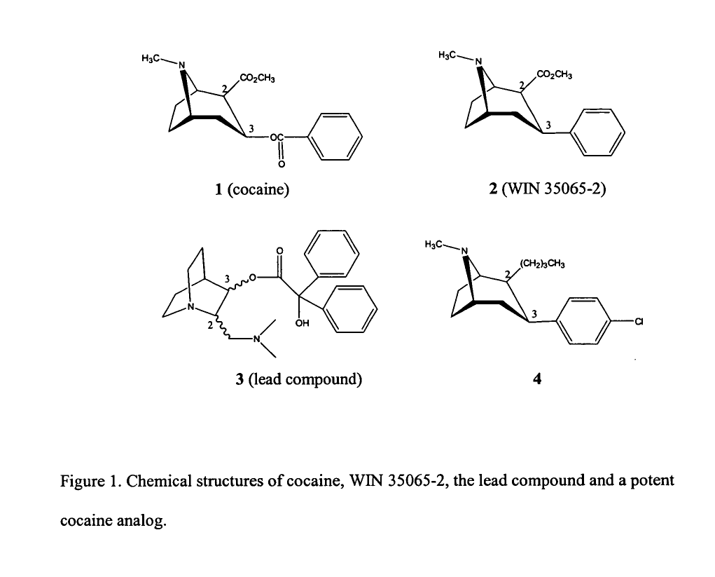

In order to design a pharmacophore representing assumable key features in DAT inhibition, a number of functional groups shared by cocaine and its analog WIN-35065 have been considered. The chemical structures of cocaine and WIN-35065 are shown in FIG. 1.

Based on extensive analysis of structure-function relationships of cocaine and its analogs, three binding elements have been identified which are believed to play important roles in the binding and reuptake activities of cocaine and its analogs, (1) an aromatic system at the 3β-position of the tropane ring; (2) a 2β ester group or a small hydrophobic group at this position; and (3) a nitrogen at position 8. The nitrogen at position 8 may be replaced by an oxygen.

The next step in formulating a pharmacophore based on the above binding elements is to determine the 3D geometric relationships of these binding elements in cocaine and its analogs and incorporating those relationships as geometric parameters, which will define the geometric requirements of the pharmacophore models.

In order to determine the geometric parameters for the design of a pharmacophore directed to cocaine based compounds, conformational analysis was performed on cocaine and WIN-35065. The X-ray crystal structure of cocaine was used as a starting point for modeling cocaine. The initial structure of WIN 35065 was built by replacing the benzoyloxy group with a phenyl group using the QUANTA molecular modeling package. The structures of both compounds were minimized, and a systematic conformational search was performed, using the program QUANTA.

The binding elements described above were represented by a nitrogen atom, a carbonyl oxygen, and an aromatic ring, respectively.

In determining the geometric requirements of the pharmacophore, three distance parameters were defined: (i) the nitrogen and the oxygen; (ii) the distance between the nitrogen and the geometric center of the aromatic ring; and (iii) the distance between the oxygen and the geometric center of the aromatic ring. The ranges for these distance parameters were determined by generating conformational profiles of cocaine and WIN-35065. The ranges were centered around the distance between two binding elements in cocaine and WIN-3 5065 conformations of low energy. The conformational profiles were then processed to determine the limits of each range.

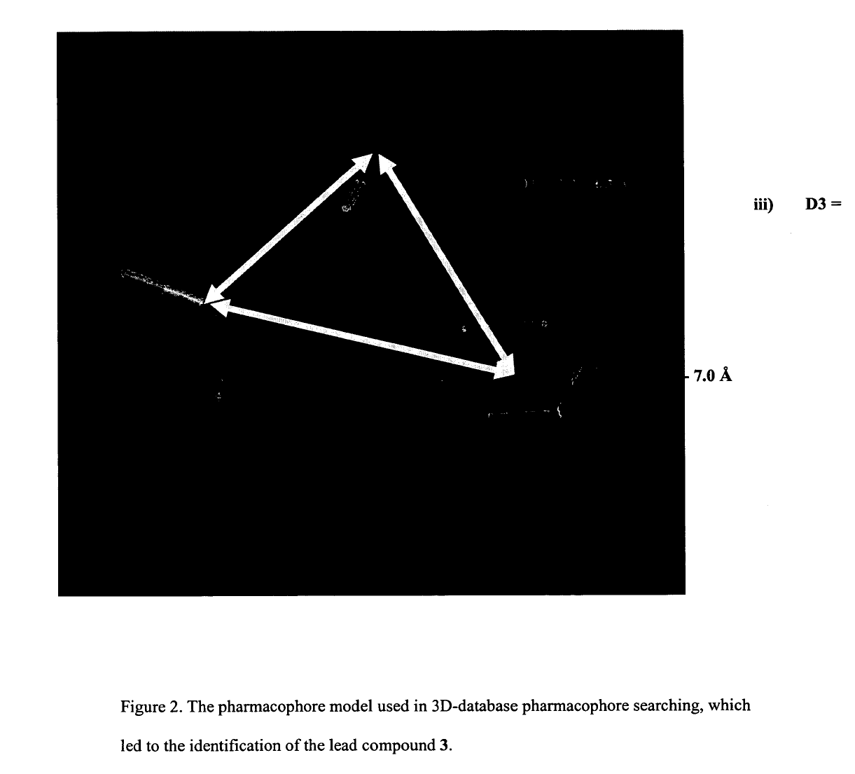

FIG. 2 shows the chemical structure and distance requirements of the pharmacophore employed in the identification of a lead compound for the design of compounds which can be useful in dopamine flow control, e.g., cocaine antagonists.

The distance requirements obtained for the pharmacophore of FIG. 2 are: (i) a distance (d1) between the nitrogen and the oxygen of from 2.2 Å to 4.5 Å; (ii) a distance (d2) between the nitrogen and the geometric center of the aromatic ring of from 5.0 Å to 7.0 Å; and (iii) a distance d3 between the oxygen and the geometric center of the aromatic ring of from 3.4 to 6.1 Å. This essentially covers the possible distance span between these atoms in cocaine and WIN 35065. Some margin was allowed for both the lowest distance value (2.6 Å) and largest distance value (4.2 Å).

The limits of the distance ranges were selected in order to provide a fairly large distance tolerance. This stems from the consideration that while the identified lead compound should be based on the general structure of cocaine, for such lead compound to be useful in the design of cocaine antagonists the distance requirements of the pharmacophore should have sufficient flexibility such that compounds having diverse chemical structures can be identified. Such a broadly defined pharmacophore allows identification of compounds that not only effectively compete with cocaine binding to the DAT, but also may display different profiles by having a binding mode significantly different from that of cocaine and WIN-35065 compounds.

3D-Database Pharmacophore Search of the NCI 3D-Databases.

As discussed above, based upon the molecular modeling studies of cocaine (1) and its WIN analogs such as 2 (WIN 35065-2), the present invention relates to the development of molecules that are designed based in the pharmacophore model described above, which includes three chemical groups believed to play an important role in binding to the DAT. It should be noted, however, that since different classes of DAT inhibitors may bind to the DAT with a combination of common and unique binding elements, more than one pharmacophore model may be developed.

Using the pharmacophore model shown in FIG. 2, we have searched 3D-databases of approximately 500,000 compounds and identified over 1000 small molecules that met the chemical and 3D geometrical requirements specified in the pharmacophore model. To date, testing of 200 potential DAT inhibitors led to the discovery of more than 20 new classes of DAT inhibitors with micromolar to nanomolar potency as measured in [³H]mazindol binding and inhibition of DA reuptake (data not shown).

Specifically, based on the pharmacophore model shown in FIG. 2, the chemical structures of the 206,876 “open” compounds in the NCI 3D-database were analyzed with the program Chem-X. During the search process, a compound was first examined for the presence of the required binding elements, i.e., a secondary or a tertiary nitrogen, a carbonyl group, and an aromatic ring system. If the three binding elements are present in a compound retrieved from the database, the program then investigates whether the compound has a conformation that meets the geometric requirements of the pharmacophore. Compounds having at least one conformation that met the distance requirements of the pharmacophore were selected for further processing. Up to 3,000,000 conformations were examined for each compound containing the three binding elements which define the pharmacophore.

Based on the pharmacophore model shown in FIG. 2, a first group of compounds containing 4094 compounds, i.e., 2% of 206,876, was formed for further processing to identify a lead compound for rational drug design.

The first step in processing the compounds in the first group involved pruning the first group by eliminating all compounds having a molecular weight greater than 1000. This is to focus the drug design on smaller compounds having a limited number of sites to be modified.

The group of compounds was further pruned by eliminating compounds wherein the nitrogen atom in the pharmacophore is not capable of accepting a hydrogen bond; e.g., due to the chemical environment of the nitrogen atom in the compound.

Finally, in order to provide a relatively small number of compounds without sacrificing the structural diversity of the group of compounds obtained through the above two pruning steps, the compounds in the pruned group were distributed in clusters according to structural similarity, each cluster providing a class of compounds represented by one compound which was selected for the next step, i.e., in vitro testing.

Based on the pruning steps described above, of the 4094 compounds identified according to the pharmacophore requirements, 385 compounds were finally selected for testing in [³H]mazindol and [³H]DA reuptake assays.

Screening of Compounds in [³H]Mazindol and [³H]DA Assays.

In the first batch of screening, 70 compounds out of the 385 selected candidates were evaluated in the [³H]mazindol binding assay. Thirteen compounds displayed more than 50% inhibition at 10 μM in the [³H]mazindol binding assay. An additional 23 compounds showed an inhibitory activity of 30% to 50% at 10 μM and 8 more compounds had an inhibitory activity of 20% to 30% at 10 μM in the [³11]mazindol binding assay. Overall, 63% of 70 (44/70) compounds showed significant activity at 10 μM in the [³H]mazindol assay. These results show that the pharmacophore model used in the 3D pharmacophore search was unexpectedly effective in identifying compounds with diverse chemical structures that can effectively compete with [³H]mazindol binding to the cocaine site on the DAT.

The group of compounds having DAT binding activity were further tested for their ability to antagonize cocaine's inhibition of [³H]DA uptake. Four classes of compounds were found to display significant functional antagonism.

In selecting lead compounds for rational drug design of novel molecules targeted at interfering with cocaine activity and DA reuptake, several approaches or strategies are adopted. One approach is based on the selection of a lead compound displaying relatively high (initial) binding affinity and inhibition of DA reuptake properties. The lead compound is then utilized in designing new molecules having binding affinity and DA reuptake properties that are significantly improved compared to the (initial) properties of the lead compound. This strategy requires that the lead compound display rather good starting or initial binding properties which are then significantly improved through rational drug design.

A second approach which is the subject of the present application is centered on the selection of a lead compound based on the degree of variation between the chemical structure of the lead compound and that of cocaine or its analogue employed in formulating the pharmacophore. That is, a lead compound having a chemical structure that is significantly different from that of cocaine is selected for further drug design even if the compound does not have a binding properties indicating strong potential in antagonizing cocaine activity as long as the compound displays some activity as cocaine antagonist. While designing novel molecules based on this approach may require more extensive research, it is believed that designing molecules having core chemical structures or scaffolds that are vastly different from those of cocaine may provide novel molecules that more potent than those designed based on lead compounds having significant cocaine antagonistic properties but also have a chemical structure that is less dissimilar to that of cocaine or its analogues employed in formulating the pharmacophore utilized in identifying or designing the lead compound.

The present invention is based on the selection of compound 3, the structure of which is shown on FIG. 1, which represents a new class of DAT inhibitors with novel structural scaffold. Compound 3, which may be classified as 2,3-disubstituted quinuclidine was found to have Kᵢ values of 7270 and 8910 nM in binding affinity and inhibition of DA reuptake, respectively, (Table 1). Despite its very weak activity, approximately 30-fold less potent than cocaine, the present invention is based on the hypothesis that Compound 3 may represent a promising lead in the design of a novel class of DAT inhibitors since it has a structural scaffold different from other classes of known DAT inhibitors. That is, the present invention is based on designing novel molecules having a chemical structure that includes the core scaffold structure of Compound 3 yet display vastly improved DAT binding affinity and DA uptake properties compared to those of Compound 3.

The subject invention is based on the discovery that rationally designed 2,3-disubstituted quinuclidines provide a novel class of dopamine transporter inhibitors. As discussed below, molecules according to the present invention having a chemical structure including the core scaffold structure associated with Compound 3 have been synthesized and tested through pharmacological testing. The molecules of the invention provide a novel class of quinuclidines that are potent DAT inhibitors. Specifically, as discussed below, one quinuclidine compound designed, synthesized and tested according to the invention has shown, in its more active enantimeric form excellent DAT binding and DA reuptake properties as illustrated by Kᵢ values of 14 and 32 nM in binding affinity and inhibition of DA reuptake, respectively.

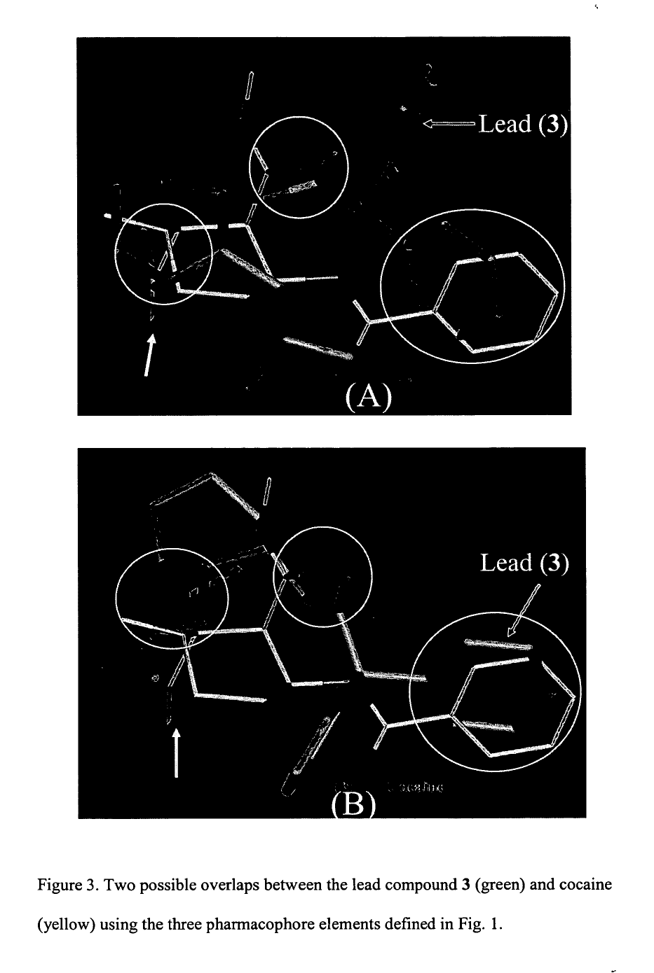

The lead compound 3 has two basic nitrogen atoms, one carbonyl group and two equivalent phenyl groups. Thus, two different overlaps are possible between Compound 3 and cocaine using the three pharmacophore elements defined in FIG. 2, i.e. a tertiary nitrogen, a carbonyl group and a phenyl ring, as the reference points. It was found that lead compound 3 has a fairly good overlap with cocaine with respect to the three crucial pharmacophore elements. The lowest root-mean-square deviation (RMSD) values in these two different overlaps (FIGS. 3 (A) and (B)) between low energy conformations of 3 and the X-ray structure of cocaine (1) are 1.12 and 0.95 Å, respectively, using the four reference points (an nitrogen atom, a carbonyl group and an aromatic ring center).

Although lead Compound 3 and cocaine have fairly good overlap with respect to the pharmacophore elements defined in FIG. 2, close examination of the two overlaps between Compound 3 and cocaine (FIG. 3) showed that there is a large exclusion volume between these two molecules. While Compound 3 and cocaine have an overlapping volume of 159 and 174 Å³ with the superposition shown in FIGS. 3 (A) and (B), respectively, they have an exclusion volume of 212 and 198 Å³, respectively.

Van der Waals (steric) interaction is perhaps the single most important factor in determining the binding mode of a drug molecule to its receptor. Thus, two compounds binding to the same binding site with similar binding modes often have a minimal exclusion volume especially if the binding site is not on the receptor surface. Molecular modeling and mutagenesis analysis showed that the binding site of cocaine at the DAT is not located on a surface. Therefore, the two overlaps shown in FIG. 3 may not represent the “true” binding mode of the lead Compound 3 in comparison to that of cocaine.

In designing more potent molecules based on Compound 3, further overlap between Compound 3 and cocaine was explored. One avenue for designing novel molecules based on Compound 3 yet have additional overlap with cocaine is based on the observation that replacement of the ester group in cocaine at position 2 with small alkyl groups results in very potent DAT inhibitors.

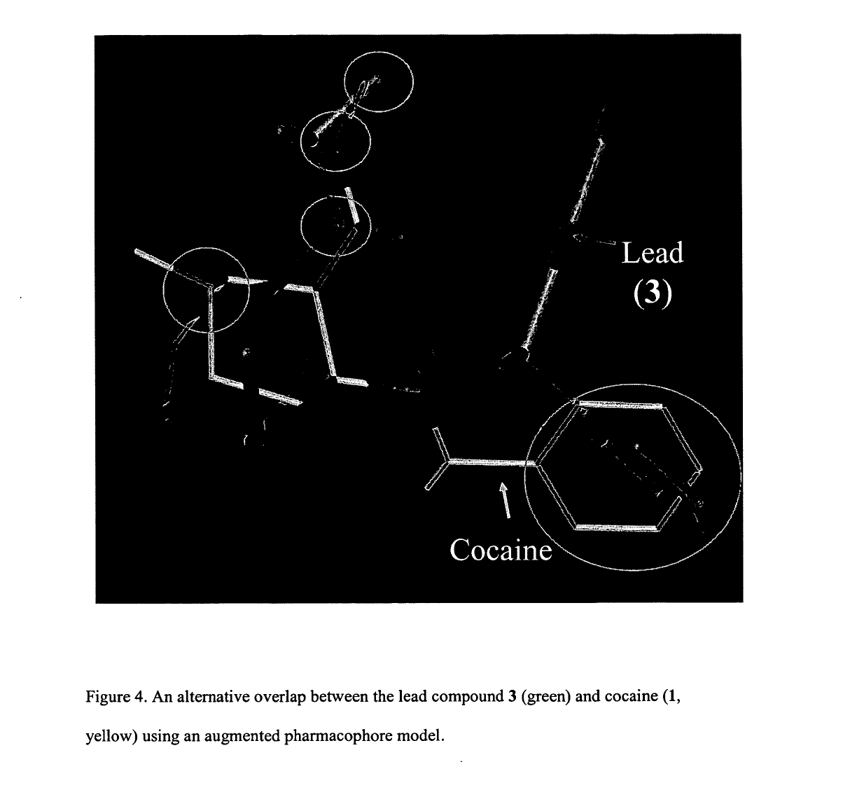

For example, Compound 4 with a butyl group at the 2□ position and a p-Cl-phenyl group at the 3□ position is a highly potent DAT inhibitor with a low nanomolar potency in binding affinity and inhibition of DA reuptake. It is believed that the carbonyl group defined in the pharmacophore model in FIG. 2 can be modified to include alkyl groups. With this modified pharmacophore model, it is hypothesized that the small N,N-dimethylmethlyamino group of Compound 3 may mimic the ester group at the 2□ position of cocaine and the 2-hydroxyl-2,2-diphenylacetate group at position 3 may mimic the benzoate group at the 3□ position of cocaine. The lowest RMSD value obtained between the low energy conformations of the lead compound 3 and the X-ray structure of cocaine is 0.50 Å, using the nitrogen in the quinuclidine ring in Compound 3 and the nitrogen in the tropane ring in cocaine, and three corresponding atoms at position 2 in the quinuclidine ring and in the tropane ring, and an aromatic ring center in Compound 3 and in cocaine as the reference points. The overlap between lead Compound 3 and cocaine is shown in FIG. 4. As can be seen, a nice overlap was found between these two molecules (FIG. 4). The 2-hydroxy-2,2-diphenylacetate group at position 3 of the quinuclidine ring locates in the same region as the phenyl ester group at the 3□ position of cocaine, and the N,N-dimethylamino group at position 2 of the quinuclidine ring overlaps nicely with the methyl ester group at the 2□ position of cocaine. However, the 2-hydroxyl-2,2-diphenylacetate group at position 3 of the quinuclidine ring appeared to be too bulky for achieving optimal potency based upon the structure-activity relationships (SAR) of cocaine and its analogs. Indeed, molecular volume calculations showed that with the overlapping manner shown in FIG. 4, Compound 3 and cocaine have an overlapping volume of 179 Å³ and an exclusion volume of 194 Å³. Although the exclusion volume is only slightly better than that shown in FIG. 3, it was found that the bulky 2-hydroxyl-2,2-diphenylacetate group accounts for much of this exclusion volume. It was shown that in cocaine, replacement of its benzoate group at the 3□ position with a phenyl group resulted in compound 2 (WIN 35065-2) with a binding affinity 4-times better than cocaine at the DAT site.

Thus, the bulky 2-hydroxy-2,2-diphenylacetate group at position 3 of the quinuclidine ring in 3 may be replaced with a simple phenyl group to improve the overlapping volume and consequently the activity. Since a small ester or a simple alkyl group at the 2□ position of cocaine is desirable for high affinity at the DAT site, the N,N-dimthylmethylamino group at position 2 of the quinuclidine ring in 3 may be replaced with a simple alkyl group for achieving potent activity at the DAT site. The two substituents at positions 2 and 3 of the quinuclidine ring can be in either trans or cis configurations. Molecular modeling showed that analogs with a cis-configuration have a better overlap with cocaine (1) and WIN 35065-2 (2).

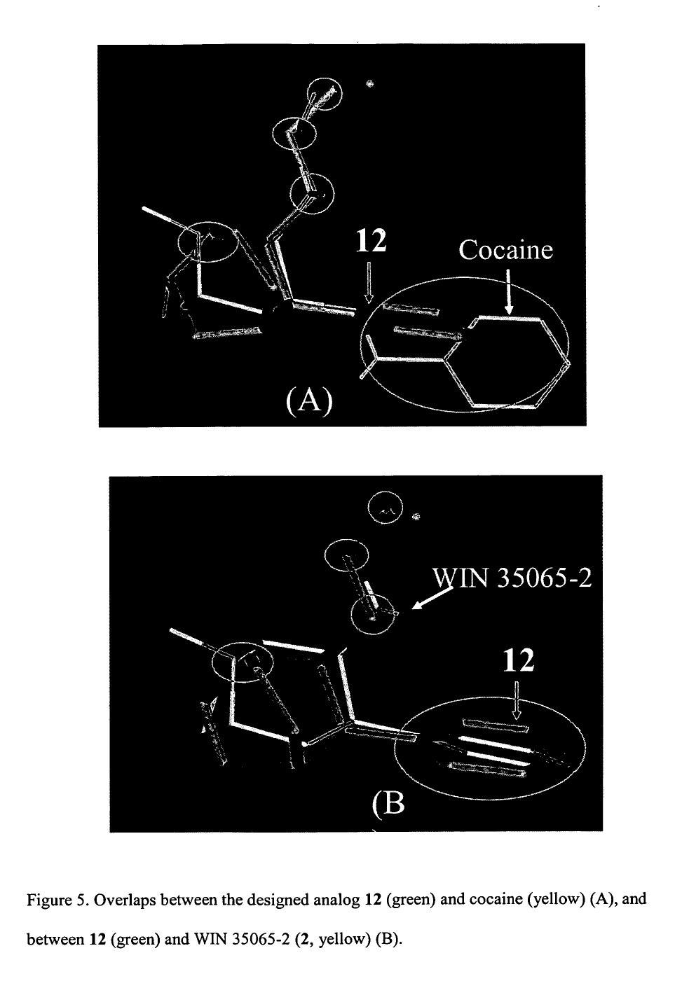

These analyses led to the design of Compound 12, which has a simple butyl group at position 2 and a phenyl group at position 3 with a cis configuration between them. A fairly good overlap was found between 12 and cocaine as depicted in FIG. 5 (A) and the lowest RMSD value was 1.07 Å using the 5 reference points shown in FIG. 5(A) with the low energy conformations of 12 and the X-ray structure of cocaine. Importantly, an excellent overlap was found between 12 and WIN 35065-2 (2), an analog more potent than cocaine, as depicted in FIG. 4(B) and the lowest RMSD value was 0.30 Å between their low energy conformations using the 5 reference points shown in FIG. 4(B) for superposition. Compound 12 and WIN 35065-2 (2) have an overlapping volume of 179 Å³ and an exclusion volume of 54 Å³, indicating an excellent overlap in terms of their overall shape. It is of interest to note that although the locations of the nitrogen atom in 12 and WIN 35065-2 (2) (FIG. 4(B)) are within 0.1 Å, the orientations of the nitrogen lone pair in these two compounds differ by approximately 60°. A previous study indicated that the orientation of the nitrogen lone pair in cocaine and its analogs is important for their selectivity among the three monoamine transporters. Taken together, our molecular modeling results suggested that 12 should be a potent DAT inhibitor.

Synthesis of 12 and other 2,3-disubstituted quinuclidines in racemic form was accomplished using a synthetic procedure as shown in Scheme I. Briefly, starting from 3-quinuclidinone (5), 2-methylene-3-quinuclidinone (6) was prepared by using Mannich reaction. Reaction of 5 with aq. dimethylamine and aq. formaldehyde in ethanol, water mixture at 70° C. gave the Mannich base, which on deamination under distillation gave compound 6 in 86% yield. Reaction of 6 with allylmagnesium bromide in the presence of CuI.Me₂S and Me₃SiCl at −78° C. furnished the conjugate addition product 7 in 47% yield along with the 1,2-addition product in 12% yield (structure not shown). Aryl Grignard addition was carried out using arylmagnesium bromide in THF at 0° C. to give compound 8, which was subsequently treated with a 1:1 mixture of EtOH and 6N HCl under reflux conditions to give the dehydrated compound 10. Reduction of the double bonds was carried out using standard hydrogenation conditions (Pd/C, H₂, EtOH, 60 Psi) to provide compound 12 in near quantitative yield.

Compound 12 was evaluated as a DAT inhibitor. Two intermediates 7 and 8 were also tested to obtain additional information about the SARs of this class of compounds. The Kᵢ values of 12 in [3H] mazindol binding and inhibition of DA reuptake are 210 and 237 nM (Table 1), respectively, representing a 31- and 32-fold improvement over the lead compound (3), and is as potent as cocaine, thus confirming our designing strategy. Compound 7 did not show any measurable activity at 10 □M in inhibition of DA reuptake (Table 1), suggesting an important role of the phenyl group and/or a detrimental effect of the ketone group at position 3. Compound 8 had a Kᵢ value of 31.2 □M (Table 1), 131-fold less potent than 12, suggesting a detrimental effect of the hydroxyl group at position 3 to the activity at the DAT site.

Previous studies have shown that an additional substitution to the phenyl ring such as a p-methyl may further improve the potency. Thus, compound 13 with an additional p-methyl group should have an improved activity if it can adopt the similar low energy conformation of 12 as shown in FIG. 4. Molecular modeling showed that 13 has an excellent overlap with WIN 35065-2 (2) and 12 with their low energy conformations. Compound 13 in racemic form was synthesized using the same procedure as for 12, as shown in Scheme I and evaluated as a DAT inhibitor. It was found that 13 has Kᵢ values of 20 and 49 nM in binding affinity and inhibition of DA reuptake, respectively, representing 365- and 181-fold improvement over the lead compound 3, and 11- and 5-fold improvement over 13 in binding and uptake activities, respectively.

To confirm the cis-configuration between substituents at positions 2 and 3 in 13 and the molecular modeling results, the X-ray structure of 13 was obtained (FIG. 5). As can be seen, the butyl group at position 2 and the p-methylphenyl at position 3 indeed have the desired cis-configuration. Since the binding of cocaine to DAT is stereospecific, it was thus interesting to investigate the stereospecificity of compound 13 in binding to the DAT. The enantiomers (+)-13 and (−)-13 were obtained using a semi-preparative chiral HPLC column (Chirex 3018), in which chiral stationary phase (CPS) consists of (S)-Proline and (R)-1-α-Naphthylethylamine covalently bound to a γ-aminopropyl silanized silica gel, and hexane/CH₂Cl₂/EtOH-TFA (20-1) in 83/15/2 ratio as the eluent. The optical rotation of (+)-13 was found to be [α]_{D}=+104 (c 0.5, acetone) and that of (−)-13 was [α]_{D}=−104 (c 0.5, acetone). It was found that (−)-13 has Kᵢ values of 14 and 32 nM, while (+)-13 has Kᵢ values of 343 and 354 nM in binding affinity and inhibition of DA reuptake, respectively. Hence, (−)-13 is approximately 2-fold more potent than (±)-13 and is 11-fold more potent than its enantiomer (+)-13.

In summary, we discovered a lead (3) through 3D-database pharmacophore searching, but its activity was approximately 30-fold less potent than cocaine in binding affinity and inhibition of DA reuptake. Molecular modeling-assisted, rational design and chemical modifications led to rapid optimization and the identification of (−)-13 with Kᵢ values of 14 and 32 nM in binding affinity and inhibition of DA reuptake, respectively, representing 519- and 278-fold improvement in binding affinity and inhibition of DA reuptake over the lead compound (3). Compound (−)-13 is 17- and 9-times more potent than cocaine in binding affinity and inhibition of DA reuptake. Previously, a class of tricyclic tropane analogs (tropaquinuclidines) was designed based upon cocaine and was shown to be potent monoamine transporter inhibitors with activity toward the serotonin and/or norepinephrine transporter. Although the quinuclidine ring in 3 (the lead compound), 12 and 13 is imbedded in the tricyclic tropaquinuclidines, the 2,3-disubstituted quinuclidines reported here differ from tropaquinuclidines in their basic ring structures and substitution patterns. Preliminary evaluations also showed that 12 and 13 have activity toward the DAT site (data not shown). Thus, compound 12 and 13 represent a novel class of potent DAT inhibitors with a basic quinuclidine ring and 2,3-disubstitutents

Chemistry:

General Methods. THF was freshly distilled under nitrogen from sodium benzophenone.

¹H and ¹³C NMR spectra were obtained with a Varian Unity Inova instrument at 300 and 75.46 MHz, respectively. ¹H chemical shifts (δ) are reported in ppm downfield from internal TMS. ¹³C chemical shifts are referenced to CDCl₃ (central peak, δ=77.0 ppm).

Melting points were determined in Pyrex capillaries with a Thomas-Hoover Unimelt apparatus and are uncorrected. Mass spectra were measured in the EI mode at an ionization potential of 70 eV. TLC was performed on Merck silica gel 60F₂₅₄ glass plates; column chromatography was performed using Merck silica gel (60-200 mesh). The following abbreviations are used: THF=tetrahydrofuran; DCM=dichloromethane; ether=diethyl ether.

2-Methylene-3-quinuclidinone (6): A solution of 3-quinuclidinone (5), (6.0 g, 48.0 mmol), 40% aqueous dimethylamine (10.0 mL, 72.0 mmol), 37% aqueous formaldehyde (6.0 mL, 72.0 mmol), 20.0 mL of ethanol and 8.0 mL of water was stirred at reflux for one hour, then at 70° C. for 17 hours and allowed to cool to room temperature. The solvents and excess reagents were evaporated in vacuo and the oily residue fractionally distilled to provide 5.7 g. (86%) of title compound as a light yellow oil, b. p. 91-92°/7 mm.

¹H NMR (300 MHz, CDCl₃) δ 1.90-1.98 (4H, m), 2.51-2.55 (1H, narrow m), 2.87-2.98 (2H, m), 3.03-3.13 (2H, m), 5.18 (1H, s), 5.78 (1H, s); ¹³C NMR (CDCl₃) δ 24.9, 40.3, 48.3, 113.3, 152.3, 204.1. Anal. (C₇H₁₁NO) C, H, N.

2-But-3-enylquinuclidin-3-one (7): To a solution of CuI.Me₂S complex [prepared by the addition of Me₂S (0.8 mL, 10.9 mmol) to CuI (1.4 g, 7.3 mmol) at 0° C.] in THF at −78° C. was added 1M solution of allylmagnesium bromide (9.5 mL) and HMPA (2.5 mL, 15.6 mmol) stirred for 20 min. To this, a mixture 2-methylene-3-quinuclidinone (6), (1.0 g, 7.3 mmol) and TMS-Cl (1.02 mL, 8.0 mmol) in THF was slowly added and stirred at the same temperature for 2 h., quenched with aq. NH₄Cl solution. The organic layer separated and the aqueous layer extracted with ethyl acetate, and the combined organic layers were dried over Na₂SO₄ and evaporated to get the crude compound. This was purified by column chromatography using ether/acetone/triethylamine in 85:10:5 ratio to afford the title compound as a colorless oil (610 mg, 47%)

¹H NMR (300 MHz, CDCl₃) δ 1.46-1.59 (1H, m), 1.79-1.93 (5H, m), 2.05-2.23 (2H, m), 2.30-2.35 (1H, m), 2.71-3.11 (5H, two m), 4.90-5.02 (2H, m), 5.68-5.82 (1H, m); ¹³C NMR (CDCl₃) δ 25.3, 26.0, 27.1, 30.4, 39.8, 40.6, 48.5, 68.8, 115.1, 137.5, 221.7; MS m/z (%) 179 (6), 110 (100); Anal. (C₁₁H₁₇NO.HCl) C, H, N.

General Procedure for the Aryl Grignard addition: To the ketone in dry THF at 0° C. was added the appropriate Grignard reagent (1.1 eq). The mixture was stirred at the same temperature for 30 min, quenched with aq. NH₄Cl, and extracted with ethyl acetate. The combined organic extracts were dried (Na₂SO₄) and concentrated under reduced pressure. The resulting crude compound was purified by column chromatography using ether/acetone/triethylamine as eluent to afford the following compounds:

2-But-3-enyl-3-phenylquinuclidin-3-ol (8): colorless thick syrup; yield 70%; ¹H NMR (300 MHz, CDCl₃) δ 1.34-1.46 (3H, m), 1.48-1.58 (1H, m), 1.81-1.94 (2H, m), 2.05-2.33 (4H, m), 2.64-2.75 (1H, m), 2.87 (2H, broad t, J=8.3 Hz), 3.10-3.20 (1H, m), 3.35-3.42 (1H, m), 4.98-5.07 (2H, m), 5.79-5.93 (1H, m), 7.30 (1H, d, J=7.3 Hz), 7.39 (2H, t, J=7.1 Hz), 7.58 (1H, d, J=7.5 Hz); ¹³C NMR (CDCl₃) δ 21.8, 23.2, 26.1, 31.4, 35.6, 41.1, 48.8, 61.8, 75.1, 114.8, 126.0, 127.2, 128.2, 138.7, 146.2; MS m/z (%) 257 (12), 124 (100); Anal. (C₁₇H₂₃NO.HCl) C, H, N.

2-But-3-enyl-3-(4-methylphenyl)quinuclidin-3-ol (9): colorless syrup; yield 74%; ¹H NMR (300 MHz, CDCl₃) δ1.32-1.54 (4H, m), 1.78-1.89 (2H, m), 2.01-2.28 (4H, two m), 2.34 (3H, s), 2.62-2.71 (1H, m), 2.80-2.86 (2H, m), 3.06-3.17 (1H, m), 3.29-3.34 (1H, m), 4.94-5.05 (2H, m), 5.76-5.89 (1H, m), 7.16 (2H, d, J=8.6 Hz), 7.42 (2H, d, J=6.6 Hz); ¹³C NMR (CDCl₃) δ 21.0, 22.0, 23.5, 26.2, 31.6, 35.8, 41.3, 49.0, 62.1, 75.1, 114.9, 126.1, 129.1, 136.9, 139.0, 143.5; Anal. (C₁₈H₂₅NO.HCl) C, H, N.

General Procedure for the dehydration: To a solution of hydroxy compound in EtOH, 6N HCl was added, refluxed overnight and cooled to room temperature. The reaction mixture was neutralized by slow addition of solid NaHCO₃ and extracted with ethyl acetate. The combined organic layers were washed with sat. NaCl solution, dried (Na₂SO₄) and concentrated to get the crude compound, which was purified by passing through a silica gel column using acetone/ether as eluent.

2-But-3-enyl-2,3-didehydro-3-phenylquinuclidine (10): colorless syrup; yield 61%; ¹H NMR (300 MHz, CDCl₃) δ 1.62-1.79 (4H, m), 2.30-2.42 (4H, m), 2.64-2.73 (2H, m), 2.86-2.92 (1H, narrow m), 3.01-3.10 (2H, m), 4.95-5.08 (2H, m), 5.78-5.90 (1H, m), 7.24-7.29 (3H, m), 7.37 (2H, t, J=7.6 Hz); ¹³C NMR (CDCl₃) δ 29.1, 30.8, 32.2, 38.8, 48.9, 114.5, 126.4, 127.6, 128.1, 138.4, 139.5, 140.2, 146.9; MS m/z (%) 239 (22), 82 (100), Anal. (C₁₇H₂₁N.HCl) C, H, N.

2-But-3-enyl-2,3-didehydro-3-(4-methylphenyl)quinuclidine (11): colorless syrup; yield 66%; ¹H NMR (300 MHz, CDCl₃) δ1.56-1.75 (4H, m), 2.28-2.38 (7H, m), 2.60-2.70 (2H, m), 2.81-2.86 (1H, m), 2.96-3.05 (2H, m), 4.90-5.05 (2H, m), 5.75-5.88 (1H, m), 7.18 (4H, s); ¹³C NMR (CDCl₃) δ 21.2, 29.4, 31.2, 32.4, 34.0, 49.2, 114.6, 127.6, 129.0, 136.1, 136.9, 138.7, 140.1, 147.0; Anal. (C₁₈H₂₃N.HCl) C, H, N.

General Procedure for the hydrogenation: A mixture of olefin and a catalytic amount of Pd/C in EtOH was hydrogenated under 60 psi of H₂ at 25° C. for 24 h. The catalyst was filtered off, and the filtrate was concentrated to give the crude compound as a yellow syrup, which on purification by column chromatography with ether/triethylamine afforded the saturated compound as a colorless thick syrup in quantitative yield.

2-Butyl-3-phenylquinuclidine (12): colorless syrup; ¹H NMR (300 MHz, CDCl₃) δ 0.80 (3H, t, J=7.1 Hz), 1.07-1.37 (6H, two m), 1.46-1.54 (1H, m), 1.70-1.76 (2H, m), 2.01-2.10 (2H, m), 2.67-2.78 (1H, m), 2.96-3.05 (1H, m), 3.09-3.29 (4H, m), 7.19-7.34 (5H, m), ¹³C NMR (CDCl₃) δ 14.0, 22.3, 22.7, 26.8, 29.7, 30.2, 30.3, 40.7, 45.4, 49.4, 60.2, 125.5, 127.8, 128.9, 142.9; MS m/z (%) 243 (18), 42 (100); Anal. (C₁₇H₂₅N.HCl) C, H, N.

2-Butyl-3-(4-methylphenyl)quinuclidine (13): colorless syrup; ¹H NMR (300 MHz, CDCl₃) δ 0.77 (3H, t, J=6.8 Hz), 1.02-1.32 (6H, two m), 1.40-1.49 (1H, m), 1.65-1.72 (2H, m), 1.96-2.06 (2H, m), 2.32 (3H, s), 2.64-2.74 (1H, m), 2.89-3.23 (5H, two m), 7.06-7.14 (4H, m); ¹³C NMR (CDCl₃) δ 14.2, 21.1, 22.5, 22.9, 27.2, 29.9, 30.4, 30.6, 40.9, 45.3, 49.7, 60.4, 128.8, 129.0, 135.1, 140.0; MS m/z (%) 257 (29), 42 (100); Anal. (C₁₈H₂₇N.HCl) C, H, N.

HPLC Separation of (±)-13

The chiral HPLC was performed on a Shimadzu SCL-10A-VP system at a flow rate of 5 mL/min at room temperature and UV detection at 254 and 280 nm. The sample for injection was prepared by dissolving racemic compound (5 mg/mL) in mobile phase and the separation was carried out by injecting 30 μL on a 250×10 mm chiral column.

Pharmacology:

[³H]Mazindol Binding

Binding assays were conducted as previously described. Briefly conventional P₂ membrane pellets were prepared by differential centrifugation from rat striatum. The P₂ pellet was resuspended in Krebs-Ringer-HEPES (KRH) buffer consisting of (in mM): NaCl (125), KCl (4.8), MgSO₄ (1.2), CaCl₂ (1.3), KH₂PO₄ (1.2), glucose (5.6), nialamide (0.01), and HEPES (25) (pH 7.4) and centrifuged again. Finally, the pellet was resuspended in 30 volumes of buffer, pelleted at 15,000×g and frozen at −80° C. until used. The striatal homogenates were thawed by resuspension in the buffer described above at 75-125 □g protein/ml and incubated with [³H]mazindol, with or without competing drugs, for 60 min in a 4° C. cold room. Non-specific binding was determined with 30 □M cocaine. The bound and free [³H]mazindol were separated by rapid vacuum filtration over Whatman GF/C filters, using a Brandel M24R cell harvester, followed by two washes with 5 ml of cold buffer. Radioactivity on the filters was then extracted by allowing to sit over night with 5 ml of scintillant. The vials were vortexed and counted. IC₅₀ values were determined using the computer program LIGAND.

Synaptosomal Uptake of [³H]DA

The effect of candidate compounds in antagonizing dopamine high-affinity uptake was determined using a method previously employed. For [³H]DA uptake, freshly dissected rat striata were homogenized with a Teflon-glass pestle in ice-cold 0.32 M sucrose and centrifuged for 10 min at 1000×g. The supernatant was centrifuged at 17,500×g for 20 min. This P₂ synaptosomal pellet was resuspended in 30 volumes of ice-cold modified KRH buffer. An aliquot of the synaptosomal suspension was preincubated with the buffer and drug for 30 min at 37° C., and uptake initiated by the addition of [³H]dopamine (5 nM, final conc.). After 5 min, uptake was terminated by adding 5 ml of cold buffer containing glucosamine as a substitute for NaCl and then finally by rapid vacuum filtration over GF-C glass fiber filters, followed by washing with two 5 ml volumes of ice-cold, sodium-free buffer. Radioactivity retained on the filters was determined by liquid scintillation spectrometry. Specific uptake is defined as that which is sensitive to inhibition by 30 □M cocaine. It is identical to that calculated by subtracting the mean of identical tubes incubated at 0° C.

IC₅₀ values were determined by a computer assisted, iterative fit to a four-parameter sigmoidal equation (ALLFIT). These values were then converted to Kᵢ values according to the Cheng-Prusoff equation assuming classical competitive inhibition. Preincubation of the drug and synaptosomes at 37° C. for 30 min was used to approximate equilibrium conditions as necessary to satisfy the requirements of the Cheng-Prusoff equation.

Molecular Modeling Studies

In Vivo Testing of Compound 6

The techniques, procedures, materials and computer programs employed in the experiments discussed herein are extensively described in the article “Discovery of a novel dopamine transporter inhibitor as a potential cocaine antagonist through 3D-data base pharmacophore searching, structure activity relationships and molecular modeling studies”, Wang et al, submitted for publication in the Journal of Medicinal Chemistry. The contents of the article and the references cited therein are hereby incorporated by reference in their entirety.

3D-Database Search

The Chem-X program (version July 96), running on a Silicon Graphics Indigo2 R10000, was used to carry out 3D-database pharmacophore searching. This program has been used to build the NCI-3D database, and was successfully used to carry out 3D-database pharmacophore searching. The primary reason for choosing this program was its ability to generate and search multiple conformations for flexible compounds in the database.

The problem of multiple conformations for flexible compounds was found to be of utmost importance m building and searching a 3D-database because flexible compounds may be able to adopt a number of different conformations depending on their environment. It is often difficult to know precisely which conformation is the biologically active one if a compound can adopt multiple conformations with little energy difference. The biologically active conformations may be different for the same compound when it binds to different receptors. Therefore, it was decided that the best way to handle this situation is to generate and search multiple conformations for flexible compounds. The ability of the Chem-X program to generate and search a large number of conformations for flexible compounds was found to be one key factor for our success in identifying a large number of structurally diverse lead compounds in several projects carried out so far.

We have found that if only single conformations for flexible compounds are searched, many identified lead compounds would be missed. Therefore, multiple conformations for flexible compounds are necessary. However, for a flexible compound with more than 10 single bonds, using a step size of 60° in generating conformations, the total number of possible conformations will exceed 60 million. In practice, we set 3 million conformations as the maximum number to be examined for any single compound.

The current version of the NCI 3D database was built using the July 94 version of the Chem-X program. It consists of 206,876 “open” compounds. Employing the Chem-X program, it is straightforward to search the NCI 3D-database of 206,876 “open” compounds for structures that meet the requirements specified in the pharmacophore models. The defined pharmacophore model was built into a pharmacophore query, which included all the specifications as described in the pharmacophore models, such as substructural requirements, and distance and distance ranges between these crucial pharmacophore components. The Chem-X program first checked if the compound has a carbonyl group, an aromatic ring, and a nitrogen attached to at least two carbon atoms and one more carbon or hydrogen. After a compound passes this sub-structural check, it was subjected to a conformational analysis. In this step, conformations were generated and evaluated with regard to geometric requirements specified in the pharmacophore query. Compounds, which have at least one conformation satisfying the geometric requirements, were considered as “hits”. “Hits” are only considered as potential candidates for biological testing. A number of additional criteria were used in the selection of compounds for biological evaluation in order to achieve maximum efficiency in the discovery of lead compounds. These criteria include simple chemical structure, small molecule, non-peptidic and chemical structure diversity.

### EXPERIMENTAL SECTION

Molecular Modeling

Conformational analysis was performed using the conformational analysis module in the QUANTA program. Generally, if a compound has fewer than five rotatable single bonds, the grid scan conformational search protocol was employed. In this protocol, each rotatable bond was systematically rotated to generate a starting conformation, which was subsequently minimized using the CHARMm program within QUANTA. If a compound has more than five rotatable bonds, a random sampling protocol was used to generate conformations. Up to 5000 conformations were generated and minimized. Energy minimization of each conformation was computed with 5000 iterations or until convergence, defined as an energy gradient of 0.001 kcal mol⁻¹ Å⁻¹ or less. An adopted basis Newton-Raphson algorithm, implemented in the CHARMm program, was used for energy minimization. A constant dielectric constant (equal to 1) was used throughout all the calculations. Upon the completion of conformation generation and energy minimization, the most stable conformation was identified (the global minimum in vacuum). It is noted, however, that the lowest energy conformation may not be the bio-active conformation, as was shown previously. For this reason, other low energy conformations, typically within 5 kcal/mol of the global minimum were identified. Cluster analysis was performed to determine the number of truly unique conformations (clusters), using the cluster analysis module available in the QUANTA program. These low energy conformational clusters together are likely to include the bio-active conformations for a compound.

3D-Database Search

The Chem-X program (version July 96), running on a Silicon Graphics Indigo2 R10000, was used to carry out 3D-database pharmacophore searching. The primary reason for choosing this program was its ability to generate and search multiple conformations for flexible compounds in the database. The problem of multiple conformations for flexible compounds was found to be important in building and searching a 3D-database because flexible compounds may be able to adopt a number of different conformations depending on their environment. It is often difficult to know precisely which conformation is the biologically active one if a compound can adopt multiple conformations with little energy difference. The biologically active conformations may be different for the same compound when it binds to different receptors. Therefore, it was decided that a best way to handle this was to generate and search multiple conformations for flexible compounds. The ability of the Chem-X program to generate and search a large number of conformations for flexible compounds was found to be one key factor for our success in identifying a large number of structurally novel, diverse lead compounds in several projects carried out so far. We have found that if only single conformations for flexible compounds are searched, many identified lead compounds would be missed. Therefore, multiple conformations for flexible compounds are necessary. However, for a flexible compound with more than 10 single bonds, using a step size of 60° in generating conformations, the total number of possible conformations will exceed 60 million. In practice, we set 3 million conformations as the maximum number to be examined for any single compound.

Employing the Chem-X program, a total of 4094 compounds were identified as “hits”, i.e. compounds that meet the requirements specified in the pharmacophore model (FIG. 1). A number of additional criteria were used in the selection of compounds for biological evaluation in order to achieve maximum efficiency in the discovery of lead compounds. These criteria include simple chemical structure, small molecular weight, non-peptidic and chemical structure diversity.

Con Formational Analysis

Conformational analysis was performed using the conformational analysis module in the QUANTA program. Generally, if a compound has fewer than five rotatable single bonds, the systematic grid conformational search protocol was employed. In this protocol, each rotatable bond was systematically rotated to generate a starting conformation, which was subsequently minimized using the CHARMm program within QUANTA. If a compound has more than five rotatable bonds, a random sampling protocol was used to generate conformations. Up to 5000 conformations were generated and minimized. Energy minimization of each conformation was computed with 5000 iterations or until convergence, defined as an energy gradient of 0.001 kcal mol⁻¹ D⁻¹ or less. An adopted basis Newton-Raphson (ABNR) algorithm, implemented in the CHARMm program, was used for energy minimization. A constant dielectric constant (equal to 1) was used throughout all the calculations. Upon the completion of conformation generation and energy minimization, the most stable conformation will be identified (the global minimum).

It is noted, however, that the lowest energy conformation may not be the bio-active conformation, as was shown previously. For this reason, other low energy conformations, typically within 5 kcal/mol of the global minimum were identified. Cluster analysis was performed to determine the number of truly unique conformations (clusters), using the cluster analysis module available in the QUANTA program. These low energy conformational clusters together are likely to include the bio-active conformations for a compound.

Synthesis of Lead Compound 3 and Its Analogs

¹H NMR and ¹³C NMR spectra were obtained with a Varian Unity Inova instrument at 300 and 75.46 MHz, respectively. ¹H chemical shifts (δ) are reported in ppm downfield from internal TMS. '³C chemical shifts are referenced to CDCl₃ (central peak, δ=77.0 ppm). NMR assignments were made with the help of COSY, DEPT, and HETCOR experiments.

Melting points were determined in Pyrex capillaries with a Thomas-.Hoover Unimelt apparatus and are uncorrected. Mass spectra were measured in the El mode at an ionization potential of 70 eV. TLC was performed on Merck silica gel 60F₂₅₄ glass plates; column chromatography was performed using Merck silica gel (60-200 mesh). The following abbreviations are used: THF=tetrahydrofuran; DCM=dichloromethane; CH₃CN=acetonitrile; ether=diethyl ether.

General Procedure for the Synthesis of Compounds 3.

In Vitro [3HlMazindol Binding Assays.

For binding assays, caudate nuclei were homogenized using a polytron in 0.32 M sucrose and centrifuged for 10 mm at 1000×g. The supernatant was resuspended in cold sucrose and centrifuged at 17,500×g for 20 mm. The pellet was resuspended in Krebs-Ringer-HEPES (KRH) buffer consisting of (in mM): NaCl (125), KCl (4.8), MgSO₄ (1.2), CaCl₂ (1.3), KH₂PO₄(1.2), glucose (5.6), nialamide (0.01), and HEPES (25) (pH 7.4) and centrifuged again. Finally, the pellet was resuspended in 30 volumes of buffer, pelleted at 15,000×g and frozen at −80° C. until used. The striatal homogenates were thawed by resuspension in the buffer described above at 75-125 μg protein/ml and incubated with [³H]mazindol, with or without competing drugs, for 60 mm in a 4° C. cold room. Non-specific binding was determined with 30 μM cocaine. The bound and free [³H]mazindol were separated by rapid vacuum filtration over Whatman GF/C filters, using a Brandel M24R cell harvester, followed by two washes with 5 ml of cold buffer. Radioactivity on the filters was then extracted by allowing to sit over night with 5 ml of scintillant. The vials were vortexed and counted. IC₅₀ values were determined using the computer program LIGAND.

Synaptosomal Uptake of [³H]Dopamine.

The effect of candidate compounds in antagonizing dopamine high-affinity uptake was determined using a method previously employed. For [³H]DA uptake, dissected rat striata were homogenized with a Teflon-glass pestle in ice-cold 0.32 M sucrose and centrifuged for 10 mm at 1000×g. The supernatant was centrifuged at 17,500×g for 20 mm. This P₂ synaptosomal pellet was resuspended in 30 volumes of ice-cold modified KRH buffer. An aliquot of the synaptosomal suspension was preincubated with the buffer and drug for 30 mm at 37° C., and uptake initiated by the addition of [³H]dopamine (5 nM, final conc.). After 5 mm, uptake was terminated by adding 5 ml of cold buffer containing glucosamine as a substitute for NaCl and then finally by rapid vacuum filtration over GF-C glass fiber filters, followed by washing with two 5 ml volumes of ice-cold, sodium-free buffer. Radioactivity retained on the filters was determined by liquid scintillation spectrometry. Specific uptake is defined as that which is sensitive to inhibition by 30 μM cocaine. It is identical to that calculated by subtracting the mean of identical tubes incubated at 0° C. [³H]5-HT and [³H]NE uptake were measured in an entirely analogous fashion using synaptosomes prepared from rat midbrain and parietal/occipital cortex, respectively. Also, specific uptake of [³H]5-HT and [³H]NE were defined in the presence of 10 uM fluoxetine and 1 uM desipramine, respectively.

Functional Antagonism Assay

First, the effects of approximate IC₁₀ to IC₅₀ concentrations of candidate of compounds on the inhibition of [³H]dopamine uptake by cocaine was determined. The IC₅₀ value of cocaine in the presence of antagonist was then compared to the IC₅₀ value of cocaine alone. Significant differences in 1C₅₀ values were compared to theoretical IC₅₀ values expected from models of “same site” antagonism. IC₅₀ values greater than those expected for “same site” antagonism will be taken as evidence of functional antagonism. Compounds demonstration antagonism were tested at lower concentrations to determine their minimum effective concentration. This test was performed under the preincubation conditions described above to allow slowly equilibrating compounds to reach equilibrium. Further, any artifactual differences in Kᵢ due to differences in temperature, buffer, etc. were negated in this assay as binding of cocaine and the putative antagonists to both the cocaine binding site and the transporter occurred under identical conditions. This insures that a right shift in the cocaine inhibition curve beyond what is expected for two drugs acting at the same site is a true measure of functional antagonism.

In Vivo Testing

Locomotor Activity Test

The test compounds were tested for the locomotor effects using male Swiss Webster mice. The potencies and efficacies [not reported] of test compounds to stimulate motor activity were determined and compared with cocaine's effects. The mice were placed in acrylic chambers which in turn were placed inside the activity monitors (Truscan, Coulbourn Instruments, Columbus, Ohio) equipped with infrared light sensitive detectors mounted along two perpendicular walls. Following 1 hr of habituation to test environment, test compounds, saline or cocaine were injected i.p. in a volume of 1 ml/100 g body weight and immediately placed back in the activity monitors. The data was recorded for a minimum of two hours. Each dose was studied in a minimum of ten mice and each mouse was used only once. The dose-effect functions on horizontal distance were constructed after subtracting the saline control group response from the test compound response. The 30-min period responses were computed from the 2 hour data. The 30-mm period during which the maximal responses would occur will be used for plotting dose-response function. Data were analyzed using standard analysis of variance and linear regression techniques. ED₅₀ values were determined from data using the linear ascending portion of the dose-effect curves.

Therapeutic Applications

Based on the results obtained with the compounds synthesized to date, it is projected that these compounds will have significant therapeutic applications. The 2,3-disubstituted quinuclidines as listed in Tables 1 and 2 were determined to be potent inhibitors for dopamine, serotonin and norepinephrine transporters. Furthermore, the selectivity of these compounds can be designed toward on particular monoamine transporter. Therefore, these compounds can be used as therapeutic agents for the treatments of a large number of neurological disorders, where blocking the uptake of the neurotransmitters and increasing the availability of the neurotransmitters can have beneficial effects. The uses of such agents are well established in the treatment of depression (Brokekkamp, C. L. E.; Leysen, D.; Peeters, B. W. M. M.; Pinder, R. M. J. Med. Chem. 1995, 38, 4615-4633), anxiety (Frances, A.; Manning, D. Marin, D. Kocsis, J.; McKinney, K.; Hall, W.; Klein, M. Psychopharamacol. suppl. 1992, 106, S82-S86), alcoholism (Kranzler, H. R.; Amine, H.; Modesto-Lowe, V.; Oncken, C. Pharmacol. Treat. Drug. Alcohol Depend. 1999, 22, 401-423), chronic pain (Sullivan, M. J. Reesor, K.; Mikail, S.; Fisher, R. Pain, 1993, 52, 294), eating disorder (Peterson, C. B.; Mitchell, J. E. J. Clin. Psychiatry, 1999, 55, 685-697), obsessive compulsive disorders (Brody, A. L. Saxena, S.; Schwartz, J. M.; Stoessel, P. W.; Maidment, K.; Phelps, M. E. Baxter, L. R. Jr. Psychiatr. Research, 1998, 84, 1-6), cocaine abuse ((a). Singh, S. Chemistry, design, and structure-activity relationship of cocaine antagonists. Chemical Reviews, 2000, 100, 925-1024 ref 7 Smith, M. P.; Hoepping, A.; Johnson, K. M.; Trzcinska, M.; Kozikowski, A. P. Dopaminergic agents for the treatment of cocaine abuse. Drug Discovery Today, 1999, 7, 322-332), and Parkinson's disease. But the present invention is not limited to these areas. Basically, the present invention is applicable to a wide range of neurological disorder, conditions or diseases where modulation of the monoamine neurotransmitter system involving dopamine (DA), serotonin (5-HT), and norepinephrine, may have beneficial effects, according to well established art in these areas.

TABLE 1
Representative monoamine transporter inhibitors of Formula (I) and their
activity at the three monoamine transporter sites.

| | | Kᵢ (nM) | | | |
|---|---|---|---|---|---|
| | | [³H] Mazin- | DA | | |
| Name | | dol Binding | Reuptake | NE | SER |
| R-cocaine | | 231 ± 22^{a} | 274 ± 20 | 108 ± 4 | 155 ± 0.4 |
| (1) | | | | | |
| Lead | | 7270 ± 400 | 8910 ± 400 | | |
| (3) | | | | | |
| 7 | 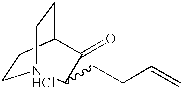 | | >10000 | | |
| 8 | 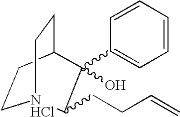 | | 31200 ± 2620 | | |
| (±)-12 | 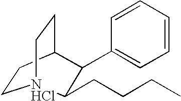 | 210 ± 17 | 237 ± 7 | 136 ± 11 | 655 ± 21 |
| (±)-13 | 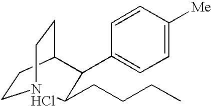 | 20 ± 1 | 49 ± 1 | 62 ± 5 | 77 ± 7 |
| (−)-13 | | 14 ± 2 | 32 ± 2 | 15 ± 3 | 26 ± 3 |
| (+)-13 | | 343 ± 16 | 354 ± 1 | 164 ± 47 | 508 ± 22 |

^{a}Mean ± standard error or range of 2-3 experiments, each conducted ussing six concentrations of drug in triplicate.

TABLE 2
Representative monoamine transporter inhibitors of Formula (II) and their
activity at the three monoamine transporter sites.

| | | Mazindol | | | |
|---|---|---|---|---|---|
| | | binding | DA | NE | SER |
| | Structure | Ki (nM) | Ki (nM) | Ki (nM) | Ki (nM) |
| 14 | 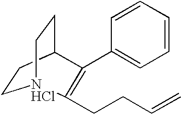 | 260 (±) 4 | 461 (±) 18 | 163 (±) 16 | 2070 (±) 230 |
| 15 | 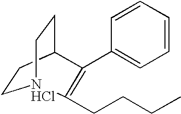 | 155 (±) 21 | 186 (±) 16 | 187 (±) 15 | 1266 (±) 158 |
| 16 | 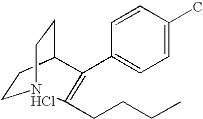 | 14 (±) 1 | 32 (±) 5 | 47 (±) 2 | 74 (±) 2 |
| 17 | 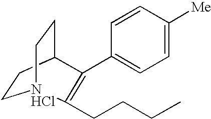 | 30 (±) 1 | 57 (±) 4 | 73 (±) 2 | 312 (±) 10 |

The subject therapies will comprise administration of at least one compound or a pharmaceutically accepted salt thereof, according to the invention in an amount sufficient to elicit a therapeutic response, e.g., inhibition of cocaine activity and/or promotion of dopamine reuptake activity in the presence of cocaine.

The compound may be administered by any pharmaceutically acceptable means, by either systemic or local administration. Suitable modes of administration include oral, dermal, e.g., via transdermal patch, inhalation, via infusion, intranasal, rectal, vaginal, topical, and parenteral (e.g., via intraperitoneal, intravenous, intramuscular, subcutaneous, injection).

Typically, oral administration or administration via injection is preferred. The subject compounds may be administered in a single dosage or chronically dependent upon the particular disease, condition of patient, toxicity of compound, and whether this compound is being utilized alone or in combination with other therapies. Chronic or repeated administration will likely be preferred based on other chemotherapies.

The subject compounds will be administered in a pharmaceutically acceptable formulation or composition. Examples of such formulations include injectable solutions, tablets, milk, or suspensions, creams, oil-in-water and water-in-oil emulsions, microcapsules and microvesicles.

These compositions will comprise conventional pharmaceutical excipients and carriers typically used in drug formulations, e.g., water, saline solutions, such as phosphate buffered saline, buffers, and surfactants.

The subject compounds may be free or entrapped in microcapsules, in colloidal drug delivery systems such as liposomes, microemulsions, and macroemulsions. Suitable materials and methods for preparing pharmaceutical formulations are disclosed in Remington's Pharmaceutical Chemistry, 16^{th} Edition, (1980). Also, solid formulations containing the subject compounds, such as tablets, and capsule formulations, may be prepared.

Suitable examples thereof include semipermeable materials of solid hydrophobic polymers containing the subject compound which may be in the form of shaped articles, e.g., films or microcapsules, as well as various other polymers and copolymers known in the art.

The dosage effective amount of compounds according to the invention will vary depending upon factors including the particular compound, toxicity, and inhibitory activity, the condition treated, and whether the compound is administered alone or with other therapies. Typically a dosage effective amount will range from about 0.0001 mg/kg to 1500 mg/kg, more preferably 1 to 1000 mg/kg, more preferably from about 1 to 150 mg/kg of body weight, and most preferably about 5 to 50 mg/kg of body weight.

The subjects treated will typically comprise mammals and most preferably will be human subjects, e.g., human cocaine addicts.

The compounds of the invention may be used alone or in combination with other agents. Additionally, the compounds may be utilized with other types of treatments to provide combination therapies which may result in synergistic results.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

While the invention has been described in terms of preferred embodiments, the skilled artisan will appreciate that various modifications, substitutions, omissions and changes may be made without departing from the spirit thereof. Accordingly, it is intended that the scope of the present invention be limited solely by the scope of the following claims, including equivalents thereof.

1. A compound or a pharmaceutically acceptable salt thereof, wherein the compound is of formulae (I) or (II): 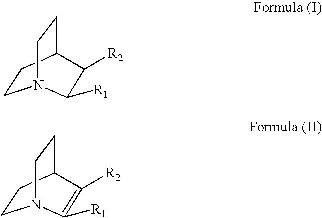
wherein R₁ is a hydrogen; linear or branched C₁-C₁₅ alkyl; C₂-C₁₅ alkenyl; C₃-C₆ cycloalkyl; mono, di, tri, tetra or penta substituted aryl or heteroaryl; —(CH₂)ₙ-aryl; COOR₃; —COO—(CH₂)ₙR₃; —(CH₂)ₙ—COOR₃; —C(O)R₃; —C(O)NHR₃; or an unsubstituted or substituted oxadiazole; R₂ is a hydrogen; linear or branched C₁-C₁₅ alkyl; C₂-C₁₅ alkenyl; C₃-C₁₀ cycloalkyl; mono, di, tri, tetra or penta substituted aryl or heteroaryl; unsubstituted or substituted naphthyl; 1,3-Benzodioxole; fluorene; indole; isoquinoline; quinoline; pyridine; pyrimidine; anthracene; or —(CH₂)ₙ-Ph; and R₃ is C₁-C₅alkyl, C₂-C₅ alkenyl, benzyl, substituted aryl or heteroaryl; and wherein R₁ and R₂ are independently selected; n=1-7, the heteroaryl comprises N, O, or S, the mono or multi substituents on the aryl or heteroaryl are independently C₁-C₅ alkyl, C₂-C₅ alkenyl, H, F, Cl, Br, I, —NO₂, NHR, or —OR, wherein R is C₁-C₇ alkyl.

2. A compound according to claim 1, wherein the compound is of formula (I) and is selected from the group consisting of the (±)-; (+)- and (−) isomers.

3. A method of preparing a compound according to claim 1, wherein the method comprises:
(a) preparing a quinuclidinone having a first substituent under Mannich reaction conditions;
(b) reacting the product of step (a) to add a second substituent to the quinuclidinone thereby producing the compound.

4. The method of claim 3, further comprising (c) reducing the compound obtained in step (b) to produce a disubstituted quinuclidine of formula (I).

5. The method of claim 4, further comprising chiral separation of the product of step (c) to obtain a compound of formula (I) in non-racemic enantiomer form.

6. The method of claim 5, wherein the chiral separation produces a (+)- enantiomer or (−)- enantimer.

7. A method of treatment of a condition or disease wherein dopamine flow in the brain plays a role, wherein the method comprises administering to a subject in need of such treatment an effective amount of a compound according to claim 1.

8. A method of treatment of a condition or disease wherein serotonin flow plays a role, wherein the method comprises administering to a subject in need of such treatment an effective amount of a compound according to claim 1.

9. A method of treatment of a condition or disease wherein norepinephrine flow in the brain plays a role, wherein the method comprises administering to a subject in need of such treatment a compound according to claim 1.

10. A method for the treatment of cocaine abuse in a subject in need of such treatment, wherein the method comprises modulating at least one of dopamine, serotonin and norepinephrine monoamine transmitter reuptake by administering to said subject a compound according to claim 1.

11. A method for the treatment of depression in a subject in need of such treatment, wherein the method comprises modulating at least one of dopamine, serotonin and norepinephrine monoamine transmitter reuptake by administering to said subject a compound according to claim 1.

12. A method for the treatment of anxiety in a subject in need of such treatment, wherein the method comprises modulating at least one of dopamine, serotonin and norepinephrine monoamine transmitter reuptake by administering to said subject a compound according to claim 1.

13. A method for the treatment of an eating disorder in a subject in need of such treatment, wherein the method comprises modulating at least one of dopamine, serotonin and norepinephrine monoamine transmitter reuptake by administering to said subject a compound according to claim 1.

14. A method for the treatment of Parkinson's disease in a subject in need of such treatment, wherein the method comprises modulating at least one of dopamine, serotonin and norepinephrine monoamine transmitter reuptake by administering to said subject a compound according to claim 1.

15. A method for the treatment of Alcoholism in a subject in need of such treatment, wherein the method comprises modulating at least one of dopamine, serotonin and norepinephrine monoamine transmitter reuptake by administering to said subject a compound according to claim 1.

16. A method for the treatment of a neurological disorder in a subject in need of such treatment, wherein the method comprises modulating at least one of dopamine, serotonin and norepinephrine monoamine transmitter reuptake by administering to said subject a compound according to claim 1.

17. A method for the treatment of chronic pain in a subject in need of such treatment, wherein the method comprises modulating at least one of dopamine, serotonin and norepinephrine monoamine transmitter reuptake by administering to said subject a compound according to claim 1.

18. A method for the treatment of obsessive compulsive disorder in a subject in need of such treatment, wherein the method comprises modulating at least one of dopamine, serotonin and norepinephrine monoamine transmitter reuptake by administering to said subject a compound according to claim 1.

19. A compound according to claim 1, wherein the compound is 2-Butyl-3-phenylquinuclidine.

20. A compound according to claim 1, wherein the compound is 2-Butyl-3-(4-methylphenyl)quinuclidine.

21. A compound according to claim 1, wherein the compound is 2-Butyl-3-(4-chlorophenyl)quinuclidine

22. The compound of claim 19, wherein the compound is in substantially pure (+)- or (−)- form.

23. The compound of claim 20, wherein the compound is in substantially pure (+)- or (−)- form.

24. A compound according to claim 1, wherein the compound is compound 16 or compound 17 as shown in Table 2.

25. The compound of claim 21 in substantially pure (+)- or (−)- form.

26. A compound according to claim 1, wherein the compound is selected from the compounds listed in Table 2.

27. A method of diagnosis of a condition wherein at least one of dopamine, serotonin and norepinephrine flow plays a role, the method comprising contacting a sample of body fluid with a compound according to claim 1, wherein the compound is labeled.

28. The method of claim 27 wherein the compound is labeled with a radioactive agent.

29. The method of claim 27, wherein the compound is labeled with a fluorescent agent.

30. The method of claim 27, wherein the compound is labeled with an electromagnetic moiety.

31. The method of claim 27, wherein the compound is conjugated to an antibody.

32. A compound according to claim 1, wherein the compound is labeled with a label selected from the group consisting of a radioactive agent and a fluorescent agent.

33. A method of treatment of a condition involving an antigen, wherein the method comprises administering to a subject a compound according to claim 1, wherein the compound is conjugated to an antibody that binds to the antigen.

34. The method of claim 33, wherein the compound of claim 1 is labeled.
